(19) United States
(12) Patent Application Publication
(10) Pub. No.: US 2013/0116781 A1
(43) Pub. Date: May 9, 2013

(54) DISCRETE PRE-ASSEMBLED MONOLITHIC AIOL ASSEMBLAGES AND AIOL ASSEMBLIES INCLUDING SAME

(71) Applicant: Joshua Ben Nun, Vitkin (IL)

(72) Inventor: Joshua Ben Nun, Vitkin (IL)

(21) Appl. No.: 13/809,483

(22) PCT Filed: Aug. 14, 2011
(86) PCT No.: PCT/IL2011/000661

§ 371 Date: Jan. 10, 2013

(30) Foreign Application Priority Data
Aug. 15, 2010 (IL) 207615
Aug. 15, 2010 (IL) 207616

(51) Int. Cl. A61F 2/16

(52) U.S. Cl. CPC A61F 2/16
USPC 623/6.43

(57) ABSTRACT

Accommodating intraocular lens (AIOL) assemblies including a discrete pre-assembled monolithic AIOL assemblage and a discrete haptics system having a haptics ring and at least two elongated C-shaped haptics for self-anchoring in a human scleral wall at the ciliary sulcus. The AIOL assemblages include an AIOL capsule and an integrally formed base member. The AIOL assemblages also include an annular haptics support surround posterior to an anterior structure on implantation in a human eye of a supine human. AIOL assemblies are assembled in situ by mounting a haptics system onto a previously implanted AIOL assemblage. The haptics system bears against the annular haptics support surround. The anterior structure is freely telescopically received in the haptics ring.

### FIELD OF THE INVENTION

The invention pertains to accommodating intraocular lenses (AIOLs) assemblies.

### BACKGROUND OF THE INVENTION

Commonly owned PCT International Application No. PCT/IL02/00693 entitled Accommodating Lens Assembly and published on 27 Feb. 2003 under PCT International Publication No. WO 03/015669 illustrates and describes accommodating intraocular lens (AIOL) assemblies, the contents of which are incorporated herein by reference. The AIOL assemblies each include a haptics system adapted to be securely fixed in a human eye's annular ciliary sulcus at at least two spaced apart stationary anchor points so that it may act as a reference plane for an AIOL of continuously variable Diopter strength affected by a human eye's capsular diaphragm under control of its sphincter-like ciliary body and acting thereagainst from a posterior direction. The haptics systems include a rigid planar haptics plate with a telescoping haptics member for sliding extension. The AIOL may not necessarily be made of a single component or material. For example, the AIOL may be in the form of a sac filled with a fluid or gel. The haptics plate and the haptics member are preferably self-anchoring as illustrated and described in commonly owned PCT International Application No. PCT/IL02/00128 entitled Intraocular Lens and published on 29 Aug. 2002 under PCT International Publication No. WO 02/065951, the contents of which are incorporated herein by reference.

Commonly owned PCT International Application No. PCT/IL2005/000456 entitled Accommodating Intraocular Lens Assemblies and Accommodation Measurement Implant and published on 10 Nov. 2005 under PCT International Publication No. WO 2005/104994 illustrates and describes AIOL assemblies enabling post implantation in situ manual selective displacement of an AIOL along a human eye's visual axis relative to at least two spaced apart stationary anchor points to a desired position to ensure that an AIOL assumes a non-compressed state in a human eye's constricted ciliary body state, the contents of which are incorporated herein by reference. WO 2005/104994's FIGS. 3 to 9 illustrate and describe a discrete haptics system selectively clamping an AIOL at a desired position along a human eye's visual axis. WO 2005/104994's FIGS. 12 to 16 illustrate and describe a haptics system with at least two haptics having radiation sensitive regions capable of undergoing plastic deformation for in situ manual displacement of an integrally formed AIOL.

Commonly owned PCT International Application No. PCT/IL2006/000406 entitled Accommodating Intraocular Lens (AIOL) Assemblies, and Discrete Components Therefor and published on 5 Oct. 2006 under PCT International Publication No. WO 2006/103674 illustrates and describes additional mechanical arrangements for achieving post implantation selective displacement of an AIOL along a human eye's visual axis, the contents are incorporated herein by reference, WO 2006/103674's FIGS. 3, 33 and 34 illustrate “push and twist” bayonet arrangements. WO 2006/103674's FIG. 35 illustrates a screw thread arrangement.

Commonly owned PCT International Application No. PCT/IL2009/000728 entitled Accommodating Intraocular Lens (AIOL) Capsules and published on 28 Jan. 2010 under PCT International Publication No. WO 2010/010565 illustrates and describes AIOL capsules filled with a transparent capsule filling constituted by either a gel or a liquid having a higher refractive index than the human eye's aqueous humor, the contents of which are incorporated herein by reference. The AIOL capsules are preferably provisioned with an integrally formed rigid haptics system for forming an unitary AIOL in a similar manner to commonly owned PCT International Application No. PCT/IL2008/000284 entitled Unitary Accommodating Intraocular Lenses (AIOLs) and Discrete Base Members For Use Therewith and published on Sep. 12, 2008 under PCT International Publication No. WO 2008/107882, the contents of which are incorporated herein by reference. Alternatively, AIOL capsules can be discrete components for in situ assembly with a discrete haptics system in a human eye thereby enabling their insertion into a human eye through a smaller corneal incision than in the case of unitary AIOL. The AIOL capsules can be fashioned for use with either a purpose designed discrete H-shaped base member or a previously implanted standard in-the-bag IOL.

Clinical practitioners implant AIOL assemblies on human patients in a supine position and are required to execute highly accurate movements in a highly confined space containing highly delicate optical structures. AIOL assemblies, namely, including an AIOL and a haptics system, completely externally assembled prior to implantation have the inherent advantage they require no in situ assembly but suffer from the disadvantage they require large corneal incisions. Against that, implantation of discrete components including an AIOL, a haptics system and possibly an additional base member for in situ assembly have the advantage they require small corneal incisions but suffer from the inherent disadvantage they complicate clinical procedures and extend procedure duration relative to completely externally assembled AIOL assemblies.

### SUMMARY OF THE INVENTION

The present invention is directed towards discrete pre-assembled monolithic AIOL assemblages including an AIOL capsule and an integrally formed base member. The discrete pre-assembled monolithic AIOL assemblages can include either an anterior or posterior bulging AIOL capsule relative to a human visual axis. The anterior or posterior bulging AIOL capsules can optionally include capsule features illustrated and described in aforesaid WO 2010/010565. The AIOL assemblages of the present invention include an annular haptics support surround posterior to an anterior structure on implantation in a human eye for affording highly convenient in situ assembly with a hitherto described discrete haptics system. Such discrete haptics systems include a haptics ring having a leading haptics ring end face, an internal peripheral haptics ring surface and a trailing haptics ring end face, and at least two elongated generally C-shaped haptics each having an attachment plate for self-anchoring in a human eye's scleral wall.

Discrete pre-assembled monolithic AIOL assemblages and discrete haptics systems are selected for in situ assembly purposes such that on mounting a haptics system onto a previously implanted AIOL assemblage, a former's trailing haptics ring end face bears against a latter's annular haptics support surround and the former's internal peripheral haptics ring surface is spaced apart from the latter's anterior structure in a plane perpendicular to the AIOL assemblage's longitudinal axis such that the anterior structure is freely telescopically received into the haptics ring which limits downward movement of the AIOL assemblage post implantation.

Thus, by virtue of the present invention precludes a clinical practitioner from having to align two AIOL components in situ with near absolute accuracy during implantation of an AIOL assembly in a human eye of a supine human patient, a clinical practitioner can more implant AIOL, assemblies of the present invention in comparison to hitherto described WO 2005/104994's clamping arrangement, and WO 2006/103674's “push and twist” bayonet arrangements and screw thread arrangement. Thus, the present invention affords AIOL assemblies requiring small corneal incisions and simplifying clinical procedures in comparison to in situ assembly of AIOL components.

This spaced apart arrangement of the present invention between a haptics ring and an AIOL assemblage's anterior structure is particularly beneficial for discrete pre-assembled monolithic AIOL assemblages with anterior bulging AIOL capsules since it allows cyclically expansion and contraction of their AIOL capsules in an outward radial direction relative to their longitudinal AIOL assemblage axis during operation of an AIOL assembly. Such cyclic movement increases mechanical efficiency of the optical power development of the anterior bulging surface of their AIOL capsules.

### BRIEF DESCRIPTION OF THE DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 corresponds to aforesaid WO 2005/104994 FIG. 3 showing an exploded perspective view of a two component AIOL assembly including a discrete AIOL and a discrete haptics system with a clamping arrangement;

FIG. 2 corresponds to aforesaid WO 2005/104994 FIG. 4 showing an assembled front view of WO 2005/104994 FIG. 3;

FIG. 3 corresponds to aforesaid WO 2006/103674 FIG. 3 showing an exploded perspective view of a two component AIOL assembly including a discrete AIOL and a discrete haptics system with a “push and twist” bayonet arrangement;

FIG. 4 corresponds to aforesaid WO 2006/103674 FIG. 35 showing an exploded perspective view of a two component AIOL assembly including a discrete AIOL and a discrete haptics system with a screw thread arrangement;

FIG. 5 corresponds to aforesaid WO 2010/010565 FIG. 3 showing an exploded view of an AIOL assembly including a haptics system, an AIOL capsule and a purpose design H-shaped base member;

FIG. 6 corresponds to aforesaid WO 2010/010565 FIG. 4 showing a front elevation view of a human eye with WO 2010/010565 FIG. 3's AIOL assembly implanted therein;

FIG. 7 corresponds to aforesaid WO 2010/010565 FIG. 5 showing an AIOL assembly including a discrete pre-assembled monolithic AIOL assemblage of an anterior bulging AIOL capsule and a haptics system, and a discrete base member;

FIG. 8 corresponds to aforesaid WO 2010/010565 FIG. 6 showing a longitudinal cross section of WO 2010/010565 FIG. 5's AIOL capsule in a non-compressed state;

FIG. 9 corresponds to aforesaid WO 2010/010565 FIG. 7 showing a longitudinal cross section of WO 2010/010565 FIG. 5's AIOL capsule in a compressed state;

FIG. 10 corresponds to aforesaid WO 2010/010565 FIG. 18 showing a side elevation view of an AIOL capsule having a capsule shell with a cone section shaped sealed cavity converging in an anterior direction along its longitudinal axis;

FIG. 11 corresponds to aforesaid WO 2010/010565 FIG. 19 showing a cross section of WO 2010/010565 FIG. 18's AIOL capsule along line E-E;

FIG. 12 corresponds to aforesaid WO 2010/010565 FIG. 12 showing a top plan view of WO 2010/010565 FIG. 3's base member;

FIG. 13 corresponds to aforesaid WO 2010/010565 FIG. 13 showing a cross section of WO 2010/010565 FIG. 12's base member along line C-C therein;

FIG. 14 corresponds to aforesaid WO 2010/010565 FIG. 14 showing deployment of the WO 2010/010565 AIOL assembly in an axial plane of a human eye in its contracted ciliary body state;

FIG. 15 corresponds to aforesaid WO 2010/010565 FIG. 15 showing deployment of the WO 2010/010565 AIOL assembly in an axial plane of a human eye in its relaxed ciliary body state;

FIG. 16 is an exploded front perspective view of a first preferred embodiment of an AIOL assembly including a discrete pre-assembled monolithic AIOL assemblage and a discrete haptics system according to the present invention;

FIG. 17 is an exploded side elevation view of FIG. 16's AIOL assembly;

FIG. 18 is a longitudinal cross section of FIG. 16's AIOL assembly in a non compressed non bulging state;

FIG. 19 is a longitudinal cross section of FIG. 16's AIOL assembly in a compressed anterior bulging state;

FIG. 20 is a longitudinal cross view of FIG. 16's AIOL assembly with a modified discrete pre-assembled monolithic AIOL assemblage;

FIG. 21 is an exploded front perspective view of a second preferred embodiment of an AIOL assembly including a discrete pre-assembled monolithic AIOL assemblage and a discrete haptics system;

FIG. 22 is a front exploded perspective view of FIG. 21's AIOL assemblage;

FIG. 23 is an exploded longitudinal cross section of FIG. 21's AIOL assemblage along line F-F in FIG. 22;

FIG. 24 is a longitudinal cross section of FIG. 21's AIOL assembly in a non-compressed non-bulging state;

FIG. 25 is a longitudinal cross section of FIG. 21's AIOL assembly in a compressed posterior bulging state;

FIG. 26 is an exploded front perspective view of a third preferred embodiment of an AIOL assembly including a discrete pre-assembled monolithic AIOL assemblage and a discrete haptics system;

FIG. 27 is an exploded front perspective view of FIG. 26's AIOL assemblage; and

FIG. 28 is a longitudinal cross section of FIG. 26's AIOL assembly along line G-G in FIG. 26 in a non-compressed non-bulging state.

### DETAILED DESCRIPTION OF PREFERRED EMBODIMENTS OF THE PRESENT INVENTION

Commonly Owned PCT International Publication No. WO 2005/104994

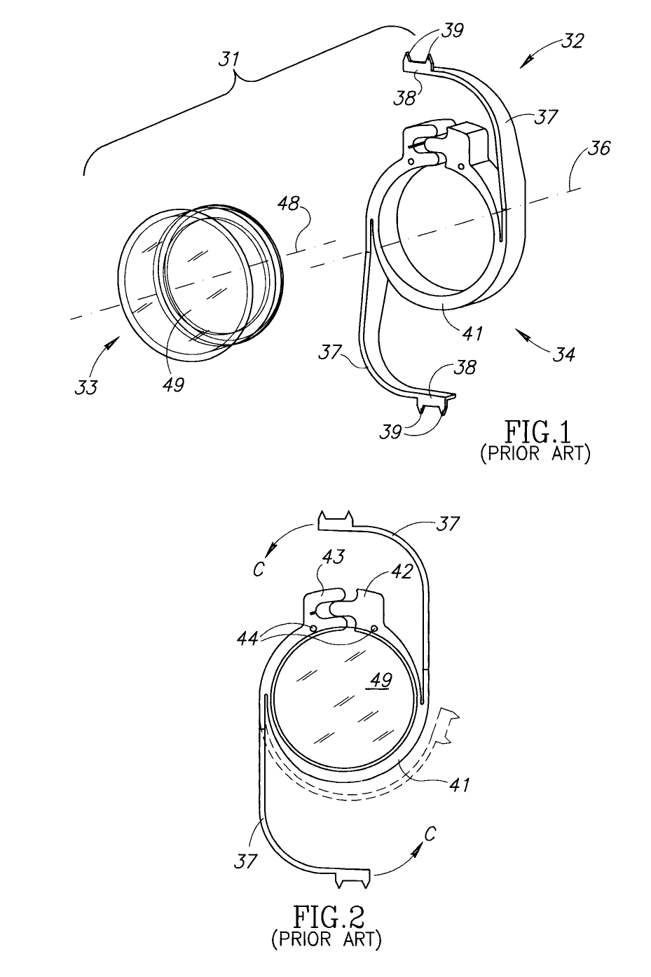

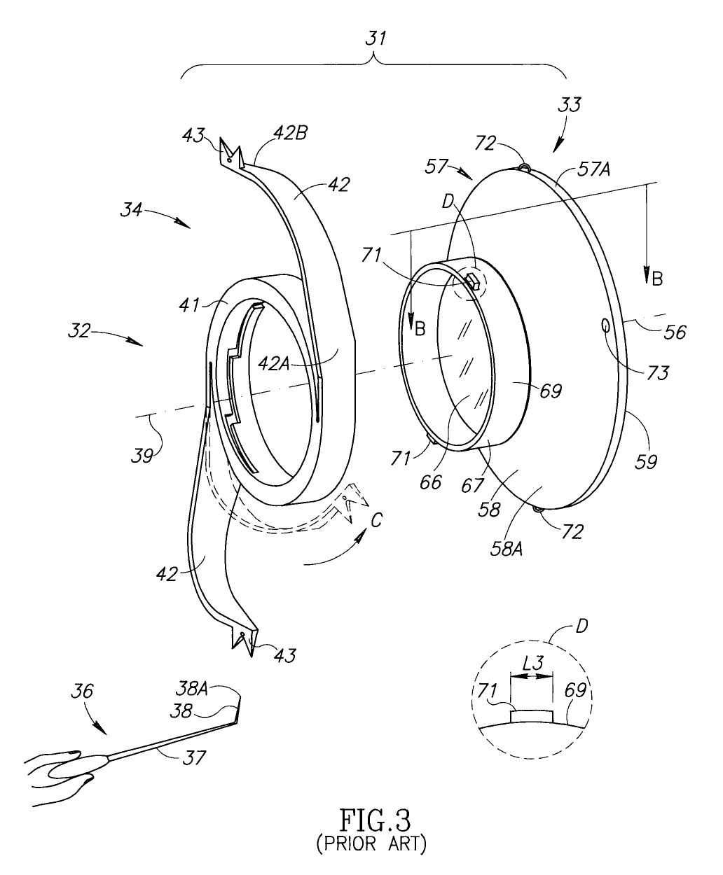

FIGS. 1 and 2 show a two component AIOL assembly 31 including a discrete haptics system 32 for selectively retaining an AIOL 33. The haptics system 32 includes a tubular main body in the form of a flexible split ring 41 with a male end 42 for releasable interference fit into a complementary female end 43 such that the main body is capable of assuming a clamp state for tightly clamping the AIOL 33 therein. The AIOL assembly 31 requires accurate co-alignment and positioning of its two discrete components.

Commonly Owned PCT International Publication No. WO 2006/103674

FIG. 3 shows a two component “push and twist” AIOL assembly 31 including a discrete haptics system 32 for selectively retaining a discrete AIOL 33 and a “push and twist” bayonet arrangement 34 requiring accurate co-alignment and positioning of the two components.

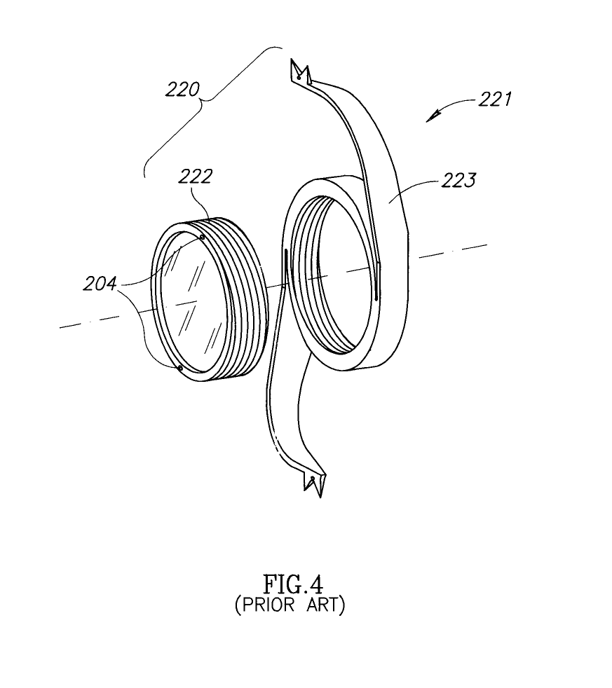

FIG. 4 shows a two component AIOL assembly 220 similar to the AIOL assembly 31 but employing a screw thread arrangement 221 instead of the “push and twist” bayonet arrangement 34.

Commonly Owned PCT International Publication No. WO 2010/010565

AIOL assemblies with a discrete pre-assembled monolithic AIOL assemblage of AIOL capsule and haptics system, and a discrete base member

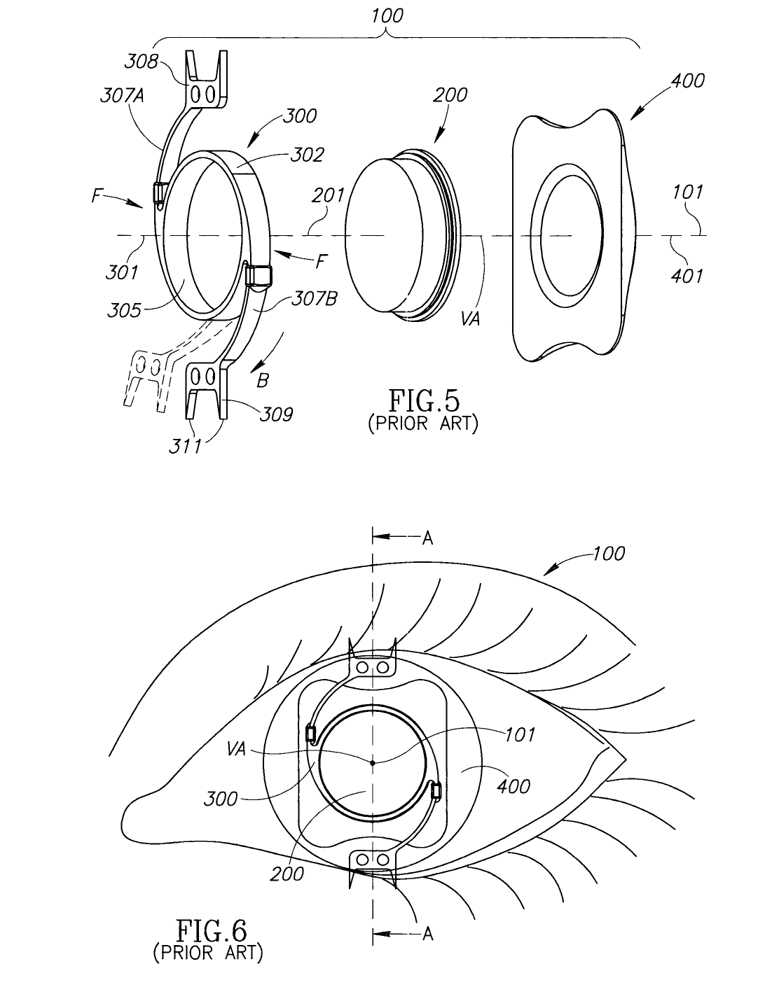

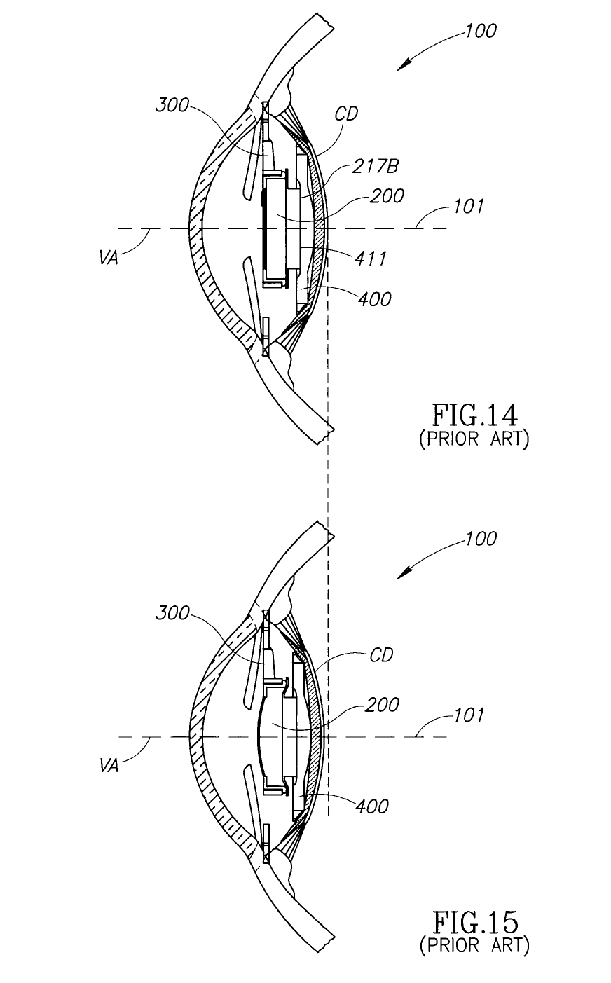

FIG. 5 shows an AIOL assembly 100 including a discrete anterior bulge AIOL capsule 200, a discrete haptics system 300 for self-anchoring in a human eye's ciliary sulcus, and a purpose designed discrete H-shaped base member 400 for interposing between the AIOL capsule 200 and a human eye's capsular diaphragm CD (see FIGS. 14 and 15) for transferring an axial compression force therefrom to the AIOL capsule 200. Alternatively, the AIOL assembly 100 can employ a standard previously implanted in the bag IOL instead of the base member 400. The AIOL assembly 100 has a longitudinal axis 101 intended to be co-directional and preferable co-axial with a human visual axis VA on implantation in a human eye. WO 2010/010565 page 9 lines 6-8 state the AIOL capsule 200 and the haptics system 300 are preferable pre-assembled using conventional assembly techniques, for example, gluing, soldering, and the like. Such pre-assembled discrete components are hereinafter referred to as a unitary AIOL 102.

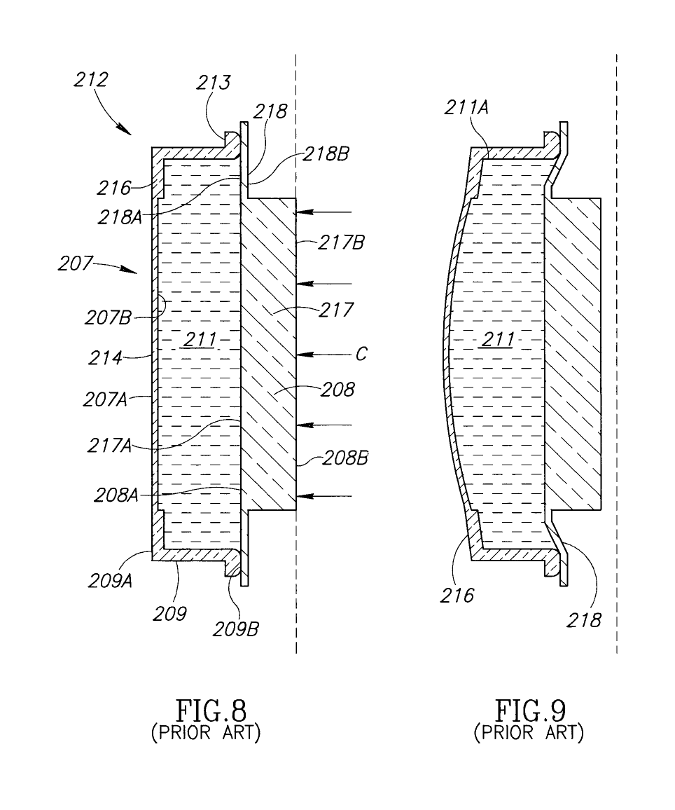

The AIOL capsule 200 includes a longitudinal axis 201 intended to be co-axial with the human visual axis VA on implantation of the AIOL assembly 100 in a human eye. The AIOL capsule 200 has a generally disc shaped resiliently elastically compressible shape memory construction. The AIOL capsule 200 includes a capsule housing 202 having an exposed leading capsule housing surface 203, an exposed trailing capsule housing surface 204 opposite and parallel to the leading capsule housing surface 203, and an external peripheral capsule housing surface 206 extending between the leading and trailing capsule housing surfaces 203 and 204. The capsule housing 202 is intended to anteriorly bulge along the human visual axis VA on application of an axial compression force against the trailing capsule housing surface 204 from a posterior direction. The AIOL capsule 200 has a continuously variable Diopter strength between a first preferably zero Diopter strength in a non-compressed state (see FIG. 8) and a second Diopter strength different than its first Diopter strength in a compressed state (see FIG. 9) on application of the axial compression force as indicated by arrows C in FIG. 8.

FIGS. 5 to 7 show the haptics system 300 includes a longitudinal haptics axis 301 intended to be co-axial with the human visual axis VA on implantation of the AIOL assembly 100 in a human eye. The haptics system 300 includes a haptics ring 302 having a leading haptics ring end face 303 defining a preferably circular aperture 304 through which the AIOL capsule 200 anteriorly bulges therethrough on application of an axial compression force from a posterior direction, an internal peripheral haptics ring surface 305 having an internal diameter ID and an opposite trailing haptics ring end face 306. The haptics system 300 is made from suitable rigid biocompatible transparent polymer material such as PMMA, and the like. The haptics ring 302 is preferable designed to squeezable on application of a pincer-like compression force denoted F in FIG. 5 such that it temporarily and reversible assumes an elliptic shape to reduce its width for lengthwise insertion through a small corneal incision to assist implantation. The internal peripheral haptics ring surface 305 is permanently attached to the external peripheral capsule housing surface 206 in the unitary AIOL 102.

The haptics system 300 includes a pair of diametrically opposite elongated generally C-shaped haptics 307A and 307B extending in opposite directions in a plane perpendicular to the longitudinal haptics axis 301. The haptics 307 terminate at a bifurcated attachment plate 308 including a pair of spaced apart puncturing members 309 having tips 311 for penetrating slightly more than half of a scleral wall's thickness of about 1 mm thereby affording anchoring points. Each haptics 307 includes a Vertical Adjustment Mechanism (VAM) for enabling in situ longitudinal displacement of the haptics ring relative to the human visual axis. WO 2010/010565 page 11 describes the VAMs with reference to WO 2010/010565 FIGS. 8 to 11 in detail.

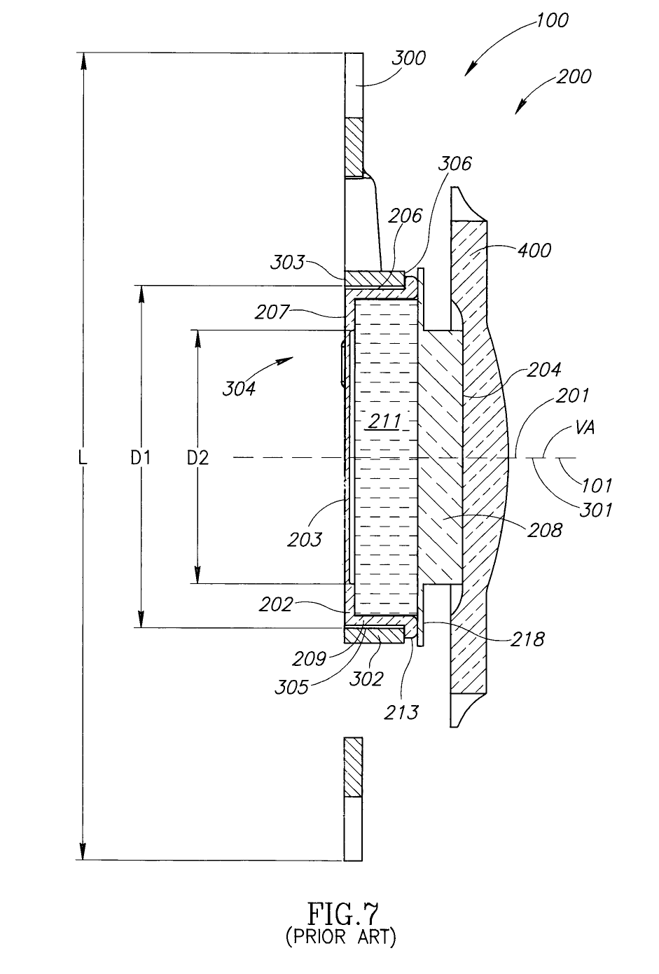

FIGS. 7 to 9 show the capsule housing 202 includes a generally circular anterior capsule plate 207 with an exposed leading capsule housing surface 207A and a concealed trailing capsule housing surface 207B, a posterior capsule plate 208 including a concealed leading surface 208A and an exposed trailing surface 208B, and a capsule ring 209 extending between the anterior and posterior capsule plates 207 and 208 and having opposite leading and trailing rims 209A and 209B. The capsule ring's leading rim 209A meets the anterior capsule plate 207 and its trailing rim 209B meets the posterior capsule plate 208. The leading surface 207A constitutes the AIOL capsule's exposed leading capsule housing surface 203, the trailing surface 208B constitutes the AIOL capsule's exposed trailing capsule housing surface 204, and the capsule ring 209 constitutes the external peripheral capsule housing surface 206. The anterior capsule plate 207, the posterior capsule plate 208 and the capsule ring 209 bound a sealed cavity 211. The anterior capsule plate 207 and the capsule ring 209 meet at a right angle affording the sealed cavity 211 with a sharp leading rim 211A.

The anterior capsule plate 207, the posterior capsule plate 208 and the capsule ring 209 are formed from biocompatible transparent polymer material. Suitable polymer materials are preferably silicon based and have a hardness rating on Shore range A between about 20 and about 80. Suitable silicon based polymer materials are commercially available from NuSil Technology LLC., US (www.nusil.com). The cavity 211 is filled with a biocompatible transparent capsule filling constituted by a gel or liquid. Suitable gels are preferably silicon based and have a hardness rating below the measurement range of Shore 00 and therefore only measurable in a penetration test using a penetrometer. Suitable silicon based gels are commercially available from NuSil Technology LLC., US (www.nusil.com).

The anterior capsule plate 207 and the capsule ring 209 are preferably manufactured as a monolithic bowl-like capsule shell 212 on which the posterior capsule plate 208 is rear sided mounted for sealing the cavity 211. The trailing rim 209B preferably extended outward to provide an annular flange 213 for permanent attachment on the trailing haptics ring end face 306 in the unitary AIOL 102. The anterior capsule plate 207 preferably includes an inner thin circular region 214 intended to undergo anterior bulging and a thicker support ring 216 connected to the capsule ring's leading rim 209A. The thin circular region 214 which acts as the AIOL capsule's main optical aperture has a diameter D2 in the region of 2.5 to 6.5 mm. The thin circular region 214 has a thickness typically in the range of 10 to 100 μm.

The posterior capsule plate 208 includes a central capsule filling displacement member 217 with a peripheral annular flange 218 intended to be rear side mounted on the annular flange 213. The capsule filling displacement member 217 and the flange 218 respectively have leading surfaces 217A and 218A constituting the posterior capsule plate's leading surface 208A. The capsule filling displacement member 217 and the peripheral annular flange 218 respectively have trailing surfaces 217B and 218B constituting the posterior capsule plate's trailing surface 208B. The flange 218 is capable of undergoing repeated back and forth flexing to enable reciprocation of the capsule filling displacement member 217 with respect to the capsule ring 209 for causing repeated anterior bulging. The capsule filling displacement member 217 and the flange 218 have co-planar leading surfaces 217A and 218A in the AIOL capsule's non-compressed state in the absence of the axial compression force C (see FIG. 8). The posterior capsule plate 208 has a stepped trailing surface 208B with the capsule filling displacement member's trailing surface 217B protruding posteriorly with respect to the flange's trailing surface 218B. The capsule filling displacement member's trailing surface 217B acts as the AIOL capsule's trailing surface 204 in terms of the axial compression force being applied thereagainst. FIG. 9 shows the support ring 216's and the flange 218's anterior flexing from their non-flexed positions on application of the axial compression force C against the capsule filling displacement member's trailing surface 217B as exemplified by the separation of the capsule filling displacement member's trailing surface 217B from the dashed reference line.

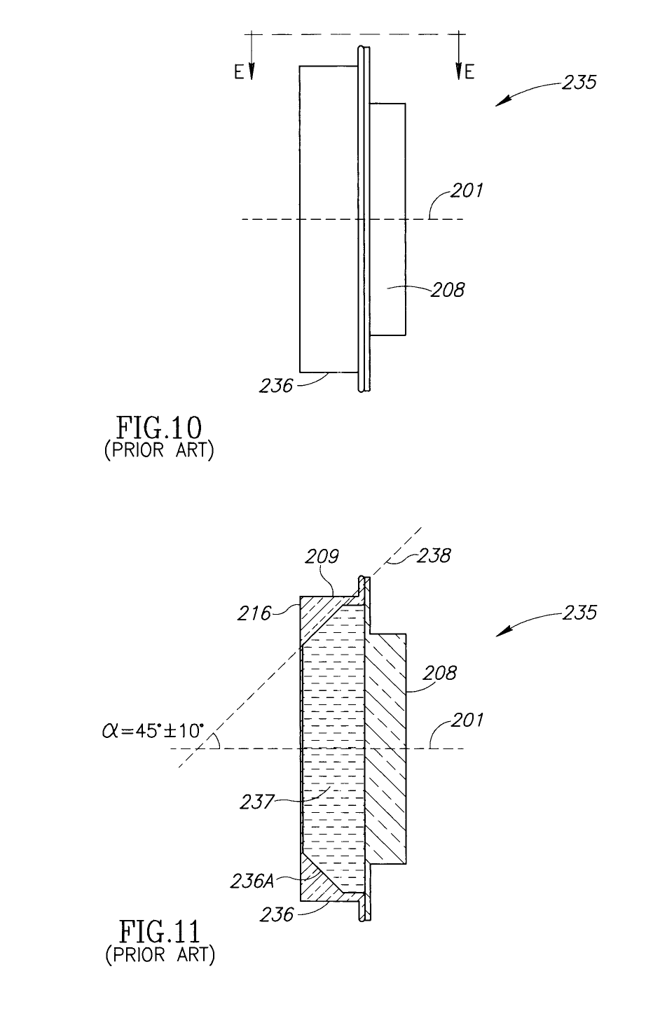

FIGS. 10 and 11 show an AIOL capsule 235 similar in construction to the AIOL capsule 200 and therefore similar parts are likewise numbered. The AIOL capsule 235 is also intended for use with the H-shaped base member 400. The AIOL capsule 235 includes a capsule shell 236 for bounding a sealed cavity 237 sealed by the posterior capsule plate 208. The capsule shell 236 has an angled leading internal surface 236A for affording the sealed cavity 237 with a cone section shape converging in an anterior direction along the longitudinal axis 201. The closed cavity 237 has a generatrix 238 defining an angle α=45°±10° with the longitudinal axis 201.

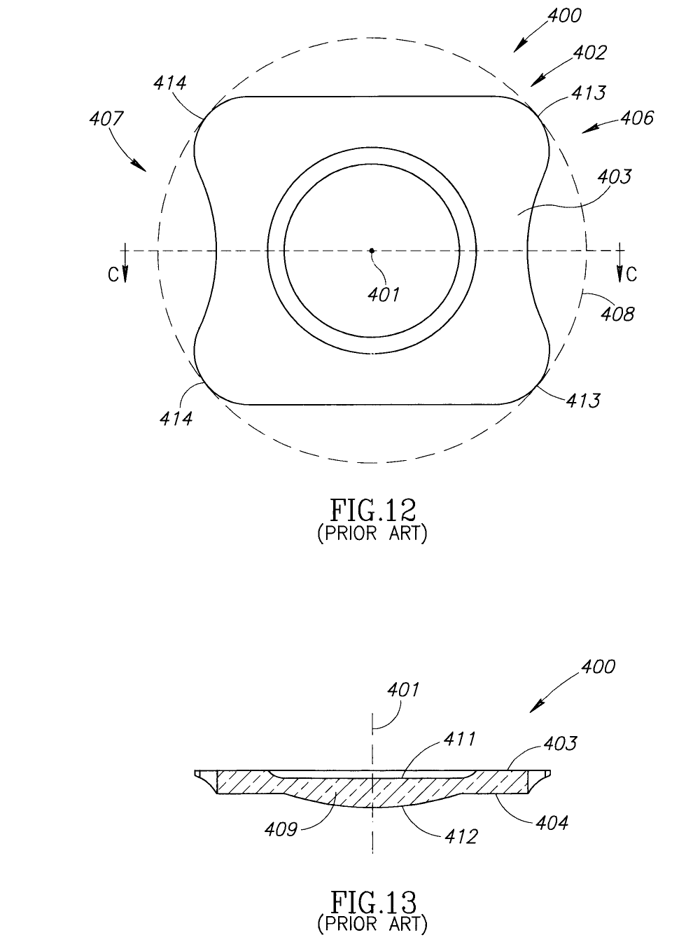

FIGS. 12 and 13 show the discrete base member 400 having a longitudinal axis 401 intended to be co-axial with the human visual axis VA on implantation in a human eye. The base member 400 has an elongated substantially planar main body 402 with opposite major anterior and posterior surfaces 403 and 404. The base member 400 is preferably made from pliable biocompatible transparent polymer material for enabling folding for insertion through a small incision into a human eye and conform to the natural curvature of a human eye's capsular diaphragm on implantation in a human eye. Suitable polymer materials include inter alia HydroxyEthylMethaAcrylate (HEMA), and the like.

The main body 402 has opposite leading and trailing ends 406 and 407 which define an imaginary circle 408 having an about 7 to 11 mm diameter which is sufficient to conform to the natural curvature of a human eye's capsular diaphragm and extend outward from a human visual axis in substantially opposite directions. The main body 402 has a central piston member 409 co-axial with the longitudinal axis 401. The central piston member 409 has leading and trailing working surfaces 411 and 412. The leading working surface 411 is preferably depressed with respect to the surrounding major anterior surface 403 thereby effectively rendering a generally circular depression for receiving the capsule filling displacement member's trailing surface 217B thereby ensuring correct alignment between the AIOL capsule 200 and the base member 400. The trailing working surface 412 is preferably shaped and dimensioned for insertion within a capsulorhexis formed in a human eye's anterior capsule. The trailing working surface 412 has a convex shape for affording upto, say, about 18 Diopter strength as required to correct a subject's vision. The leading end 406 has a first pair of spaced apart lateral wings 413 and the trailing end 407 has an opposite pair of spaced apart lateral wings 414 extending radial from the piston member 409 thereby affording an overall H-shape to the base member 400 in FIG. 6's top plan view.

FIGS. 14 and 15 show deployment of the WO 2010/010565 AIOL assembly respectively in its contracted ciliary body state and relaxed ciliary body state.

AIOL Assemblies with a Discrete Pre-Assembled Monolithic AIOL Assemblage of AIOL Capsule and Base Member, and a Discrete Haptics System

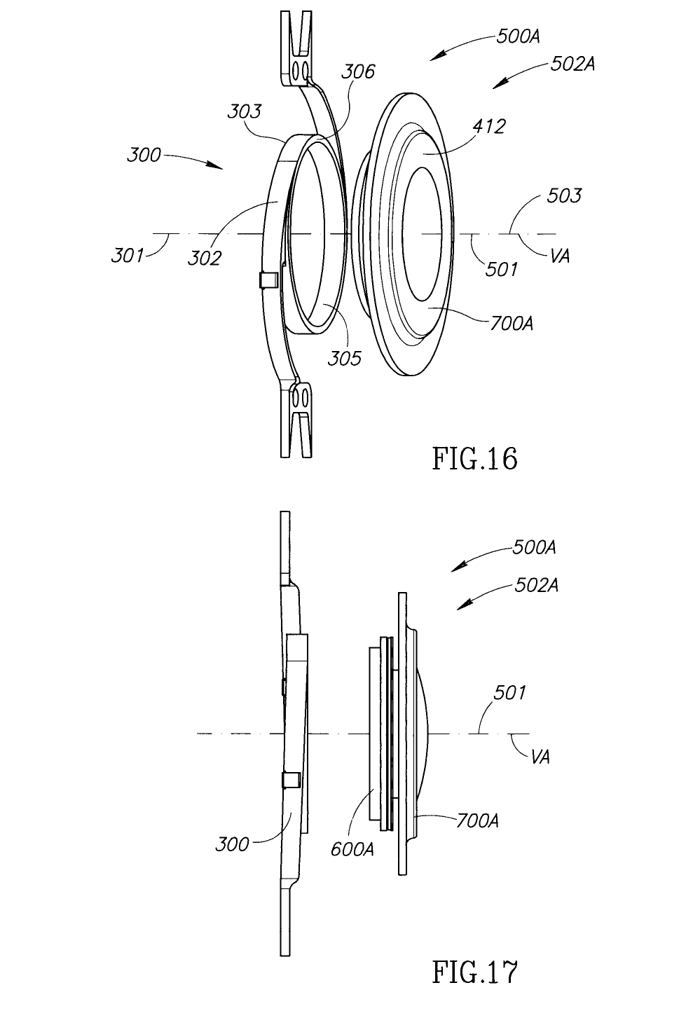

FIGS. 16 to 19 show an AIOL assembly 500A similar to the AIOL assembly 100 and therefore similar parts are likewise numbered. The AIOL assembly 500A has a longitudinal assembly axis 501 and includes a discrete pre-assembled monolithic AIOL assemblage 502A and a discrete haptics system 300 for self-anchoring in a human eye's ciliary sulcus. The discrete pre-assembled monolithic AIOL assemblage 502A has a longitudinal AIOL assemblage axis 503 intended to be co-directional and preferably co-axial with a human visual axis. The AIOL assemblage 502A includes an anterior bulge AIOL capsule 600A anterior to an integrally formed base member 700A for interposing between the AIOL capsule 600A and a human eye's capsular diaphragm for transferring an axial compression force therefrom to the AIOL capsule 600A. Thus, the AIOL assembly 500A has an effectively reverse construction to the AIOL assembly 100 insofar that its discrete pre-assembled monolithic AIOL assemblage 502A is integrally formed with the base member 700A instead of the haptics system 300.

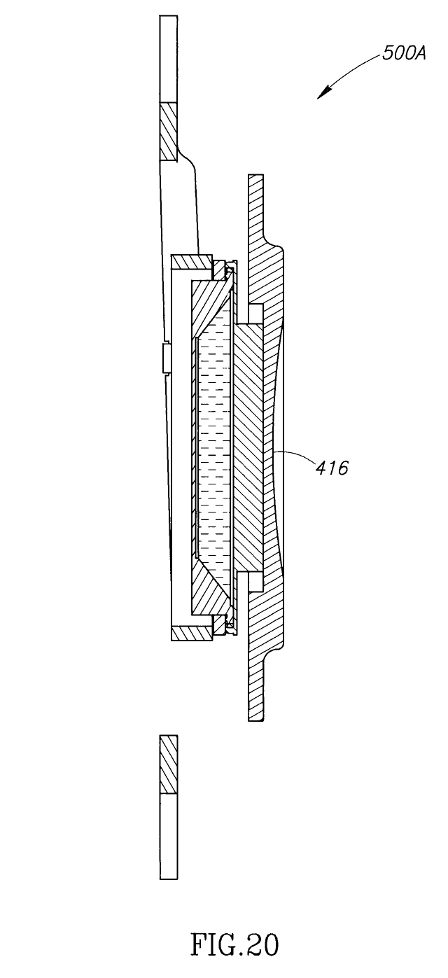

The AIOL capsule 600A has the same basic construction as the AIOL capsule 235 and therefore similar parts are likewise numbered. The AIOL capsule 600A includes a sealed cavity 211 filled with a biocompatible transparent capsule filling constituted by a gel or liquid and a capsule filling displacement member 217 for selective displacement into the sealed cavity 211 to causing the AIOL capsule 600A to bulge. The sealed cavity 211 has a cone shaped shape converging in an anterior direction. The generatrix can define an angle α=45°±25° with the longitudinal capsule axis. The base member 700A has the same basic construction as the base member 400 and therefore similar parts are likewise numbered. The AIOL capsule 600A's trailing surface 204 is permanently attached to the base member 700A's leading working surface 411 by suitable assembly techniques such as gluing, soldering, and the like. The AIOL capsule 600A includes an annular support 601 mounted on the annular flange 213 and the posterior capsule plate's peripheral annular flange 218 having an anterior directed rim 218A. The trailing working surface 412 can be alternatively formed with a concave surface 416 having a Diopter strength of about −10 for correcting a subject's vision (see FIG. 20).

For the purpose of in situ assembly of the discrete pre-assembled monolithic AIOL assemblage 502A and the discrete haptics system 300 to form the AIOL assembly 500A, the annular support 601 acts as an annular haptics support surround 504 posterior to an anterior structure 506 constituted by the leading section of the AIOL capsule 600A on implantation of the AIOL assemblage 502A against a capsular diaphragm. Accordingly, the external peripheral capsule housing surface 206 constitutes an external peripheral anterior structure surface having an external diameter ED.

The discrete pre-assembled monolithic AIOL assemblage 502A and the discrete haptics system 300 are selected such that on mounting the haptics system 300 onto the previously implanted AIOL assemblage 502A from an anterior direction, the trailing haptics ring end face 306 bears against the haptics support surround 504 to urge the AIOL assemblage 502A against the capsular diaphragm and the anterior structure 506, namely, the AIOL capsule 502A's leading section, is spaced apart from the internal peripheral haptics ring surface 305 in a plane perpendicular to the longitudinal AIOL assemblage axis 503 such that it is freely telescopically received in the haptics ring 302.

Both the internal peripheral haptics ring surface 305 and the external peripheral anterior structure surface 206 are right cylindrical surfaces with respect to their respective longitudinal haptics axis 301 and longitudinal AIOL assemblage axis 503 such that the internal peripheral haptics ring surface 305 bounds a generally cylindrical separation 507 with the external peripheral capsule housing surface 206. The diameter difference ID-ED is in the range of from about 100 μm up to about 500 μm.

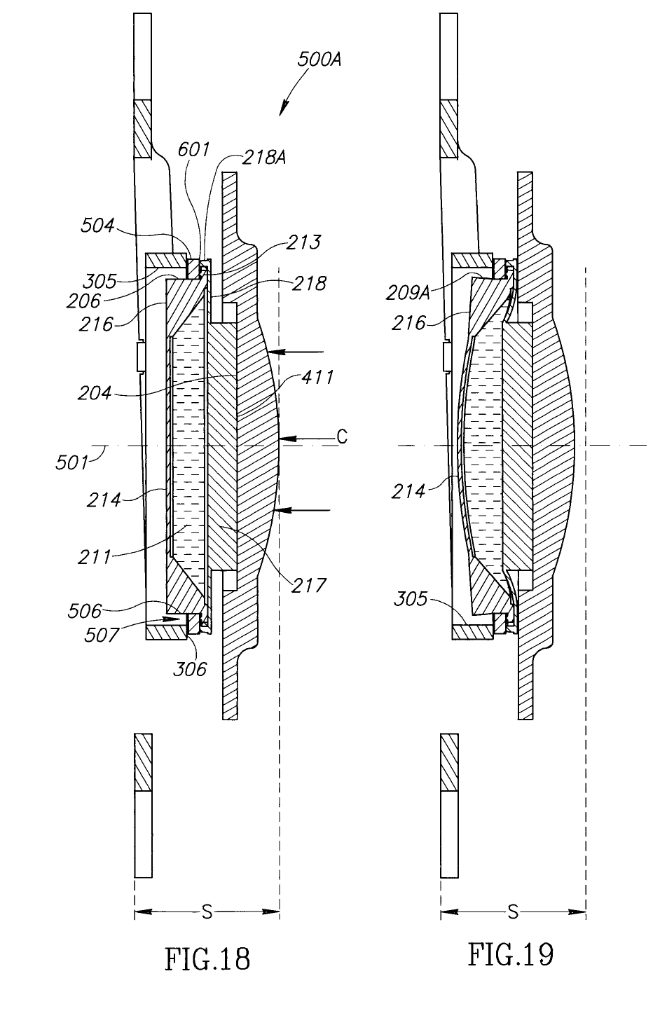

The AIOL assemblage 502A has a non-compressed non-bulging state in a human eye's contracted ciliary body state (see FIG. 18) and a compressed anterior bulging state in a human eye's relaxed ciliary body state (see FIG. 19). FIG. 18 shows the inner thin circular region 214 flush with the support ring 216 whilst FIG. 19 shows the AIOL assemblage 502A has been urged in an anterior direction towards the haptics system 300 as evidenced by a greater separation between the base member 700A and the dashed reference line positioned at the same separation S from the stationary haptics system 300 as in FIG. 18.

The generally cylindrical separation 507 also enables slight radial outwards expansion of the AIOL capsule 600A with respect to the longitudinal AIOL assemblage axis 503 during operation of the AIOL assembly 500A as evidenced by the leading rim 209A having a smaller separation from the inside peripheral haptics ring surface 305 in FIG. 19 than in FIG. 18. Such cyclic movement increases mechanical efficiency of the optical power development of the anterior bulging surface of the AIOL capsule 600 thereby facilitating AIOL assembly 500A operation.

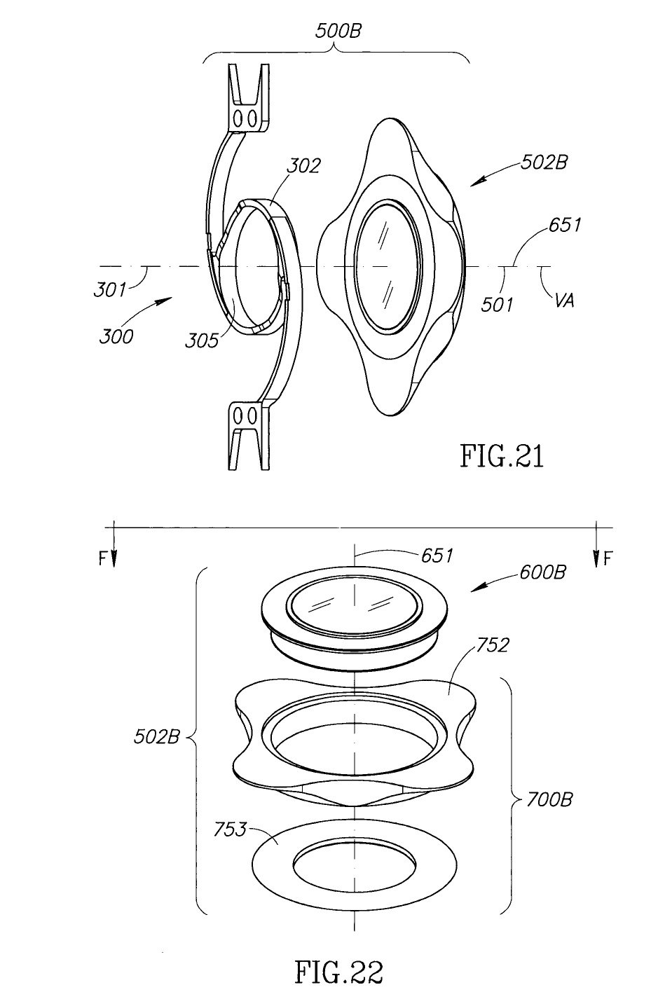

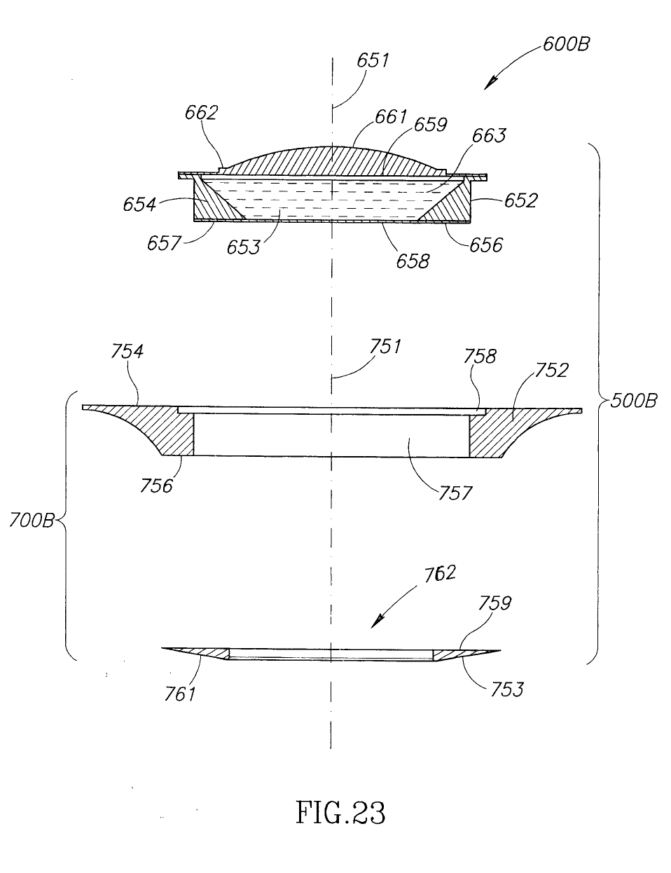

FIGS. 21 to 25 show an AIOL assembly 500B similar to the AIOL assembly 500A and therefore similar parts are likewise numbered. The former 500B differs from the latter 500A insofar as the former 500B includes a discrete pre-assembled monolithic AIOL assemblage 502B including a posterior bulging AIOL capsule 600B and an integrally formed base member 700B. The discrete pre-assembled monolithic AIOL assemblage 502B has a longitudinal AIOL assemblage axis 503 intended to be co-directional and preferably co-axial with a human visual axis.

The AIOL capsule 600B has a longitudinal capsule axis 651 coaxial with the longitudinal AIOL assemblage axis 503 and a capsule shell 652 similar to the capsule shell 235 for bounding a sealed cavity 653 having a cone section shape converging in a posterior direction along the longitudinal capsule axis 651 on implantation in a human eye. The AIOL capsule 600B includes a capsule ring 654 and a posterior capsule plate 656 having an annular surround 657 rear side mounted on the capsule ring 654 and an inner thin circular region 658 intended to undergo repeated cyclic posterior bulging and unbulging. The AIOL capsule 600B is sealed by an anterior capsule plate 659 having a central anterior lens 661 having an anterior lens peripheral rim 662. The anterior lens 661 acts as a capsule filling displacement member for selective displacement into the sealed cavity 653 to causing the AIOL capsule 600B to bulge. The anterior lens 661 can be either convex as shown or concave for affording Diopter strength to correct a subject's vision for distance vision. The anterior capsule plate 659 has a diameter greater than the anterior lens peripheral rim 662's diameter to bound a thin flexible annulus 663 capable of undergoing repeated back and forth flexing to enable reciprocation of the anterior lens 661 relative to the capsule ring 654.

The base member 700B has a longitudinal base member axis 751 co-axial with longitudinal AIOL assemblage axis 503 and a two piece construction including an anterior annular planar main body 752 intended to snugly and fixedly receive the AIOL capsule 600B therein, and a posterior planar bulge protection member 753. The main body 752 has an about 7 to 11 mm diameter which is sufficient to conform to the natural curvature of a human eye's capsular diaphragm. The main body 752 has opposite major anterior and posterior surfaces 754 and 756 and a central throughgoing bore 757 shaped and dimensioned for snugly and fixedly receiving the capsule ring 654 therein. The major anterior surface 754 is provisioned with a shallow annular recess 758 shaped and dimensioned for snugly and fixedly receiving the anterior capsule plate 659 therein.

The bulge protection member 753 has opposite anterior and posterior major surface 759 and 761 and is formed with a central throughgoing bore 762 into which the AIOL capsule 600B bulges thereinto. The throughgoing bore 762 has a larger diameter than the inner thin circular region 658's diameter to avoid the bulge protection member 753 interfering with posterior bulging. The anterior major surface 759 is fixedly mounted on the major posterior surface 756. The posterior major surface 761 is inclined in a posterior direction for contoured abutment against a human eye's capsular diaphragm.

For the purpose of in situ assembly of the discrete pre-assembled monolithic AIOL assemblage 602A and a discrete haptics system 300 to form the AIOL assembly 600A, the anterior lens peripheral rim 662 acts as an annular haptics support surround 504 posterior to an anterior structure 506 constituted by the anterior lens 661 on implantation of the AIOL assemblage 602A against a capsular diaphragm.

The discrete pre-assembled monolithic AIOL assemblage 602A and the discrete haptics system 300 are selected such that on mounting the haptics system 300 onto the previously implanted AIOL assemblage 602A from an anterior direction, the trailing haptics ring end face 306 bears against the haptics support surround 504 to urge the AIOL assemblage 602A against the capsular diaphragm and the anterior structure 506, namely, the anterior lens 661, is spaced apart from the internal peripheral haptics ring surface 305 in a plane perpendicular to the longitudinal AIOL assemblage axis 503 such that it is freely telescopically received in the haptics ring 302.

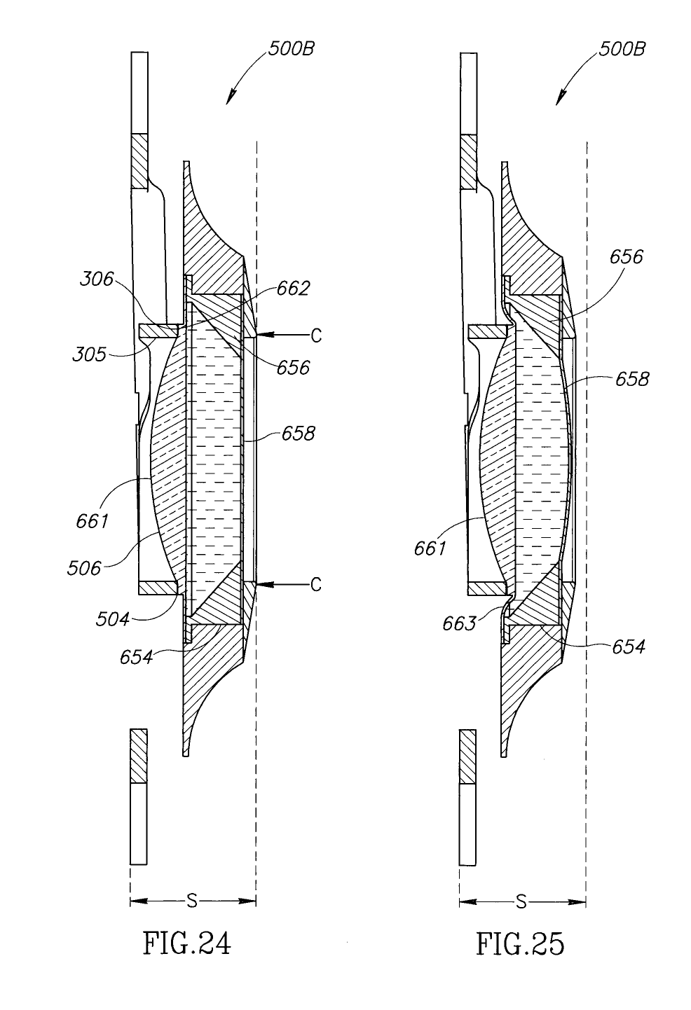

The AIOL assemblage 502B has a non-compressed non-bulging state in a human eye's contracted ciliary body state (see FIG. 24) and a compressed posterior bulging state in a human eye's relaxed ciliary body state (see FIG. 25). FIG. 24 shows the anterior lens 661 anterior to the capsule ring 654 and the inner thin circular region 658 flush with the annular surround 656. FIG. 25 shows the AIOL assemblage 502B has been urged in an anterior direction towards the stationary haptics system 300 as evidenced by a greater separation between the base member 700B and the dashed reference line positioned at the same separation S from the stationary haptics system 300 as in FIG. 24. FIG. 25 shows the central lens 661 acting as a capsule filling displacement member depressed into the capsule ring 654 and the inner thin circular region 658 posterior bulging with respect to the annular surround 656. The relative movement of the anterior lens 661 with respect to the capsule ring 654 is achieved by the flexing of the flexible annulus 663.

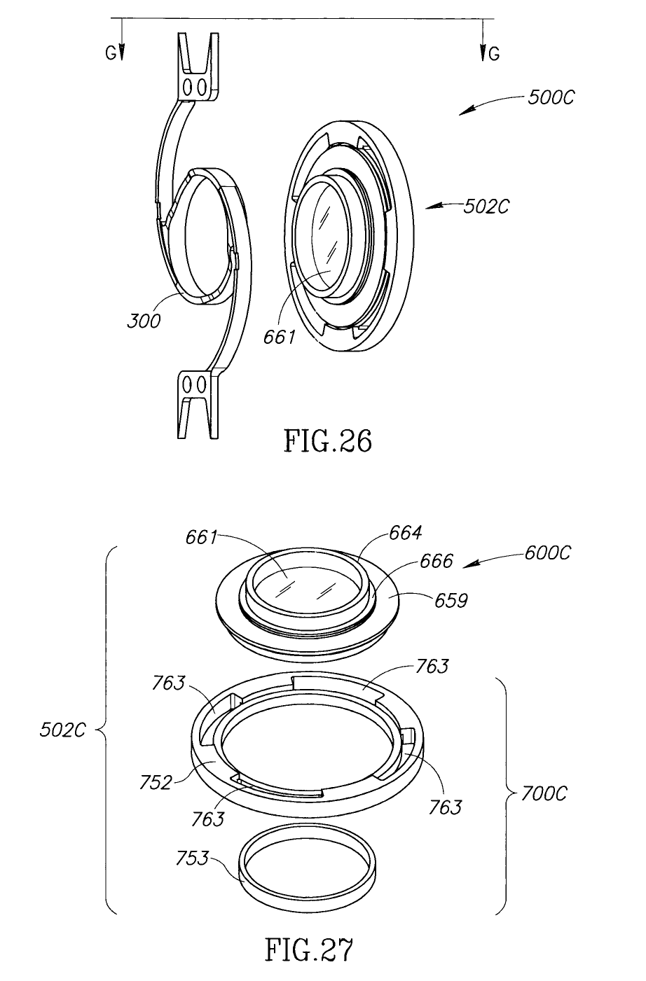

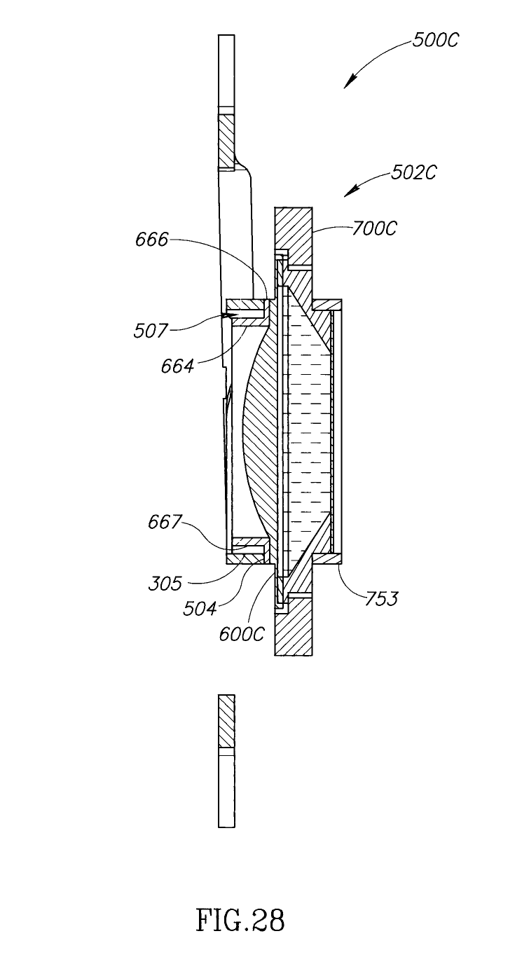

FIGS. 26 to 28 show an AIOL assembly 500C with a discrete pre-assembled monolithic AIOL assemblage 502C similar in construction and operation as the pre-assembled AIOL assemblage 502B and therefore similar parts are likewise numbered. The former 502C differs from the latter 502B as follows: First, the anterior capsule plate 659 includes a fixedly mounted ring 664 surrounding the anterior lens 661. The ring 664 has a posterior flange 666 against which the trailing haptics ring end face 306 bears against on implantation of the haptics system 300 subsequent to implantation of the AIOL assemblage 502C and an external peripheral ring surface 667. Thus, the posterior flange 666 acts as the annular haptics support surround 504 instead of the anterior lens peripheral rim 662. The ring 664 is spaced apart from the internal peripheral haptics ring surface 305 such that it bounds a generally cylindrical separation 507 with the external peripheral ring surface 667 similar to the AIOL assembly 500A. Second, the main body 752 is formed with two pairs of opposite throughgoing slots 763 for facilitating sideways compression to form an oval shape for implantation through a small incision. And third, an annular bulge protection member 753 shaped and dimensioned for insertion within a capsulorhexis formed in a human eye's anterior capsule.

The implantation of AIOL assemblies 500 is as follows:

A clinical practitioner implants a discrete pre-assembled monolithic AIOL assemblage 502 through a corneal incision into a subject's eye and places its base member 700 on the subject's capsular diaphragm and aligns the longitudinal AIOL assemblage axis 503 with his human visual axis. The clinical practitioner implants a haptics system 300 through the corneal incision and anchors its leading haptics 307A into the far side of the subject's eye's scleral wall. The clinical practitioner manipulates the haptics system 300 to mount its haptics ring 302 onto the AIOL assemblage 502. The clinical practitioner manipulates the trailing haptics 307B into the subject's eye through the corneal incision for anchoring into the near side of the subject's eye's scleral wall opposite the leading haptics 307A.

While the invention has been described with respect to a limited number of embodiments, it will be appreciated that many variations, modifications, and other applications of the invention can be made within the scope of the appended claims.

1. A discrete pre-assembled monolithic AIOL assemblage for implantation in a human eye of a supine human, the human eye having a human visual axis, a sclera wall of tough connective tissue, an annular ciliary sulcus, and a sphincter-like ciliary body for tensioning a capsular diaphragm in an anterior direction along the human visual axis on its relaxation from a contracted ciliary body state to a relaxed ciliary body state,
the AIOL assemblage for use with a discrete haptics system having a longitudinal haptics axis for co-axial alignment with the human visual axis and a haptics ring with at least two elongated generally C-shaped haptics extending therefrom in opposite directions in a plane perpendicular to said longitudinal haptics axis,
each haptics terminating in an attachment plate having at least one pointed puncturing member for penetrating the tough connective tissue of the scleral wall for self-anchoring implantation at at least two spaced apart stationary anchor points in the sclera wall at the annular ciliary sulcus,
said haptics ring having a leading haptics ring end face, an internal peripheral haptics ring surface and a trailing haptics ring end face opposite said leading haptics ring end face,
the AIOL assemblage having a longitudinal AIOL assemblage axis for co-axial alignment with the human visual axis on implantation in the human eye, said AIOL assemblage comprising:
(a) an AIOL capsule having a sealed cavity filled with a transparent capsule filling and a capsule filling displacement member for selective displacement into said sealed cavity for causing said AIOL capsule to bulge,
(b) a base member integrally formed with said AIOL capsule and interdisposed between said AIOL capsule and the capsular diaphragm for bearing against the capsular diaphragm for applying an axial compression force from a posterior direction to said AIOL capsule,
said AIOL capsule bulging along the human visual axis on application of said axial compression force and reverting to its non-flexed position in the absence of said compression force, whereby said AIOL capsule has a continuously variable Diopter strength ranging between a first Diopter strength in a non-compressed state and a second Diopter strength different than said first Diopter strength in a compressed state on application of said axial compression force,
the AIOL assemblage including an annular haptics support surround posterior to an anterior structure on implantation of the AIOL assemblage in the human eye against the capsular diaphragm such that on mounting the haptics system onto the previously implanted AIOL assemblage from an anterior direction, its trailing haptics ring end face bears against said annular haptics support surround to urge the AIOL assemblage against the capsular diaphragm and the internal peripheral haptics ring surface is spaced apart from said anterior structure in a plane perpendicular to said longitudinal AIOL assemblage axis such that said anterior structure is freely telescopically received into the haptics ring.

2. The AIOL assemblage according to claim 1 wherein the internal peripheral haptics ring surface has an internal diameter ID and said anterior structure has an external peripheral anterior structure surface with an external diameter ED such that the diameter difference ID-ED is in the range of from about 100 μm up to about 500 μm.

3. The AIOL assemblage according to claim 2 wherein said internal peripheral haptics ring surface and said external peripheral anterior structure surface are generally right cylindrical surfaces with respect to their respective longitudinal haptics axis and longitudinal AIOL assemblage axis such that they bound a generally cylindrical separation on anterior mounting the haptics system on the previously implanted AIOL assemblage.

4. The AIOL assemblage according to claim 1 wherein said AIOL capsule bulges in an anterior direction along the human visual axis on application of said axial compression force.

5. The AIOL assemblage according to claim 1 wherein said AIOL capsule bulges in a posterior direction along the human visual axis on application of said axial compression force.

6. The AIOL assemblage according to claim 1 wherein said anterior structure is constituted by said AIOL capsule.

7. The AIOL assemblage according to claim 1 wherein said anterior structure is constituted by an anterior lens mounted on said AIOL capsule.

8. An AIOL assembly for implantation in a human eye of a supine human, the human eye having a human visual axis, a sclera wall of tough connective tissue, an annular ciliary sulcus, and a sphincter-like ciliary body for tensioning a capsular diaphragm in an anterior direction along the human visual axis on its relaxation from a contracted ciliary body state to a relaxed ciliary body state, the AIOL assembly comprising:
(a) a discrete pre-assembled monolithic AIOL assemblage according to claim 1; and
(b) a discrete haptics system having a longitudinal haptics axis for co-axial alignment with the human visual axis and a haptics ring with at least two elongated generally C-shaped haptics extending therefrom in opposite directions in a plane perpendicular to said longitudinal haptics axis,
each haptics terminating in an attachment plate having at least one pointed puncturing member for penetrating the tough connective tissue of the human eye's sclera for self-anchoring implantation at at least two spaced apart stationary anchor points in the sclera wall at the annular ciliary sulcus,
said haptics ring having a leading haptics ring end face, an internal peripheral haptics ring surface and a trailing haptics ring end face opposite said leading haptics ring end face such that on mounting the haptics system onto a previously implanted AIOL assemblage from an anterior direction, said trailing haptics ring end face bears against said annular haptics support surround to urge said AIOL assemblage against the capsular diaphragm and said internal peripheral haptics
